(19) United States
(12) Patent Application Publication
(10) Pub. No.: US 2006/0134235 A1
(43) Pub. Date: Jun. 22, 2006

(54) External preparation for improving blood flow

(75) Inventors: Kinya Takagaki, Fukuoka (JP); Takeshi Mitsui, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(73) Assignee: Toyo Shinyaku Co., Ltd.

(21) Appl. No.: 11/020,693

(22) Filed: Dec. 22, 2004

(51) Int. Cl. A61K 36/899; A61K 31/35; A61K 36/45; A61K 36/16; A61K 36/48; A61K 36/73; A61K 36/13

(52) U.S. Cl. 424/732; 424/765; 424/752; 424/750; 424/757; 424/770; 514/27; 514/456

(57) ABSTRACT

The present invention provides an external preparation for improving blood flow that comprises proanthocyanidins. The external preparation for improving blood flow provides an excellent effect of improving blood flow. When the proanthocyanidins that contain preferably at least 20 wt % OPCs are employed, a further excellent effect of improving blood flow can be achieved.

### BACKGROUND OF THE INVENTION

1. Field of the Invention

The present invention relates to an external preparation for improving blood flow that comprises proanthocyanidins. More specifically, the present invention relates to an external preparation for skin that, when applied to the skin or the mucous membrane, promotes blood circulation in the area of application.

2. Description of the Related Art

Poor blood circulation in the skin is regarded as a cause of skin problems such as dullness and unevenness of skin color, and furthermore, as a cause of chilblains and the condition of being overly sensitive to the cold. As therapeutic agents for these diseases, ointments, creams, and the like that contain an oil, a humectant, or the like that is derived from a plant and less irritating to the skin have been extensively studied.

As substances for promoting blood circulation that can be used for these therapeutic agents, for example, plant extracts, such as oils and fats obtained from the seeds of shea tree, nicotinic acid derivatives, vitamin E derivatives, and swertia herb extract, are known (e.g., Japanese Patent No. 3113705 and Japanese Laid-Open Patent Publication No. 11-269054).

However, the conventional external preparations for improving blood flow have disadvantages such as poor retention of their effects. Moreover, active components in the external preparations that can be derived from plants are not clearly identified. Thus, even if extracts are obtained from the same plant, the resultant effects of these extracts often vary from each other. Furthermore, the usage of a chemical substance such as vitamin E for an external preparation can be limited due to the specific characteristics of the chemical substance.

Therefore, there is a demand for an external preparation for skin that provides a superior effect of improving blood flow.

### SUMMARY OF THE INVENTION

The inventors of the present invention performed in-depth research on the external preparation for improving blood flow in view of the above-described problems and found that, surprisingly, a superior effect of improving blood flow is obtained by applying proanthocyanidins to the skin, the mucous membrane, and the like, and thus, achieved the present invention.

The present invention provides an external preparation for improving blood flow that comprises a proanthocyanidin.

In one embodiment, the proanthocyanidin is derived from a bark of pine; a fruit or seeds of grape, blueberry, strawberry, avocado, locust, or cowberry; barley; wheat; soybean; black soybean; cacao; an inner skin of peanuts; or leaves of ginkgo.

In a preferred embodiment, the proanthocyanidin comprises at least 20 wt % of oligomeric proanthocyanidin.

In a more preferred embodiment, the external preparation for improving blood flow further comprises a component providing an effect of improving blood flow and/or a plant extract providing the effect of improving blood flow, wherein the component and the plant extract do not contain the proanthocyanidin.

According to the present invention, an external preparation for improving blood flow that contains proanthocyanidins is provided. The external preparation for improving blood flow of the present invention provides an effect of improving subcutaneous blood circulation and the like. The proanthocyanidins that are contained in the external preparation for improving blood flow of the present invention provide a better effect of improving blood flow than conventional plant extracts such as swertia herb extract do, and also the resultant effect continues for a longer period of time. Furthermore, when the proanthocyanidins contain at least 20 wt % of OPCs, a further superior effect of improving blood flow can be achieved.

### BRIEF DESCRIPTION OF THE DRAWINGS

FIG. 1 is a graph showing a change in blood flow improvement rate over time after application of 0.1 mL of an external preparation for improving blood flow of the present invention.

FIG. 2 is a graph showing a change in blood flow improvement rate over time after application of 0.03 mL of the external preparation for improving blood flow of the present invention.

### DESCRIPTION OF THE PREFERRED EMBODIMENT

Hereinafter, the external preparation for improving blood flow of the present invention will be described. It should be noted that the following description is not limiting the present invention, and it is apparent to those skilled in the art that various alternations can be made within the scope of the spirit of the present invention.

In the present invention, proanthocyanidins refer to a group of compounds that are condensation products having flavan-3-ol and/or flavan-3,4-diol as a constituent unit and having a degree of polymerization of 2 or more, and proanthocyanidins also include extracts derived from raw material plants containing these compounds.

As the proanthocyanidins that are used for the external preparation for improving blood flow of the present invention, proanthocyanidins containing a large amount of condensation products having a low degree of polymerization are preferably used. As the condensation products having a low degree of polymerization, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer) are even more preferable.

In this specification, among proanthocyanidins, condensation products having flavan-3-ol and/or flavan-3,4-diol as a constituent unit and having a degree of polymerization of 2 to 4 are referred to as oligomeric proanthocyanidins (OPCs). OPCs, which are one type of polyphenol, are potent antioxidants produced by plants, and contained concentratedly in portions of plant leaves, bark, or skin or seeds of fruits. More specifically, they are contained in the bark of pine; the fruit or seeds of grape, blueberry, strawberry, avocado, locust, and cowberry; barley; wheat; soybean; black soybean; cacao; the inner skin of peanuts; and the leaves of ginkgo, for example. Moreover, it is known that OPCs are also contained in cola nuts in West Africa; the roots of Rathania in Peru; and Japanese green tea. OPCs are substances which cannot be produced in the human body. Proanthocyanidins containing a large amount of OPCs are preferred. Among these, in particular, it is preferable to use a pine bark extract. Among proanthocyanidins, OPCs are especially abundant in pine bark, and thus, the pine bark extract is preferably used for the proanthocyanidins in the present invention.

When proanthocyanidins having a high OPC content are used, a better effect of improving blood flow can be achieved than in the case where proanthocyanidins having a high degree of polymerization (having a low OPC content) are used.

Hereinafter, a method for preparing proanthocyanidins will be described taking a pine bark extract that contains OPCs abundantly as an example.

As the pine bark extract, an extract from the bark of plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins from the flavonoid family, which are the main component of the French maritime pine bark, have a potent antioxidation ability of removing active oxygen.

The pine bark extract is obtained by extracting the bark of the above-described pines using water or an organic solvent. When using water, it is preferable to employ warm water or hot water. In order to increase the extraction efficiency, it is preferable to add a salt such as sodium chloride to the water. As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. The water and the organic solvents may be used alone or in combination. In particular, hot water, aqueous ethanol, and aqueous propylene glycol are preferably used.

The method for extracting proanthocyanidins from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V % to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and examples thereof include a batch method using liquid carbon dioxide, a reflux method using liquid carbon dioxide, and a reflux method using supercritical carbon dioxide.

It is also possible to employ a combination of a plurality of extraction processes to perform extraction from pine bark. By combining a plurality of extraction processes, pine bark extracts with various components can be obtained.

The pine bark extract that is used for the external preparation for improving blood flow of the present invention is specifically prepared using the following method. However, this method is merely an example, and the pine bark extract used for the present invention is not limited to the extract obtained by this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of ⅕ of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs that have a degree of polymerization of 2 to 4 and at least 5 wt % of catechins can be obtained.

It is preferable that an extract derived from a raw material plant, such as a pine bark extract, contains at least 40 wt % of proanthocyanidins. Furthermore, the OPC content in this extract derived from a raw material plant is preferably at least 20 wt % and more preferably at least 40 wt %.

An extract derived from the raw material plant contains catechins as well as OPCs. The term “Catechins” is a general term referring to polyhydroxyflavan-3-ols. As the catechins, for example, (+)-catechin, (−)-epicatechin, (+)-gallocatechin, (−)-epigallocatechin, epigallocatechin gallate, and epicatechin gallate are known. Gallocatechin, afzelechin, and 3-galloyl derivatives of (+)-catechin or gallocatechin are isolated from natural products, in addition to (+)-catechin that is called catechin in a narrow sense. Catechins are known to have a cancer inhibiting ability, an arteriosclerosis preventing ability, a lipid metabolism disorder inhibiting ability, a blood pressure elevation inhibiting ability, a thrombosis preventing ability, an antiallergic ability, an antiviral ability, an antibacterial ability, a dental caries preventing ability, a halitosis preventing ability, an intestinal flora normalization ability, an active oxygen or free radical eliminating ability, an antioxidation ability, and the like. Moreover, catechins are known to have an antidiabetic ability of inhibiting an elevation of blood glucose. Furthermore, catechins have the property of both increasing the solubility in water and being activated in the presence of OPCs.

It is preferable that catechins are contained in the above-described extract derived from a raw material plant in a ratio of 5 wt % or more. Alternatively, it is also preferable that a formulation is prepared so that it contains a raw material plant extract containing at least 20 wt % of OPCs and furthermore, contains catechins in a ratio of 5 wt % or more. For example, when the catechin content in a pine bark extract is less than 5 wt %, it is possible to add catechins so that the catechin content becomes at least 5 wt %. It is most preferable to use a pine bark extract containing at least 5 wt % of catechins and at least 20 wt % of OPCs.

The proanthocyanidin content in the external preparation for improving blood flow of the present invention is not particularly limited, but it is preferably 0.0001 wt % to 5 wt %, more preferably 0.001 wt % to 2 wt %, and even more preferably 0.01 wt % to 1 wt %.

When an extract derived from a raw material plant that contains proanthocyanidins is employed, the amount of proanthocyanidins can be calculated based on the proanthocyanidin content in the extract. As such extract derived from a raw material plant, a pine bark extract is preferable.

When the thus obtained external preparation for improving blood flow of the present invention is applied to the skin or the mucous membrane, it promotes blood circulation in the area of application. The proanthocyanidins contained in the external preparation for improving blood flow of the present invention provide an excellent effect of improving blood flow, and the effect is superior to that derived from conventional plant extracts such as swertia herb extract. Also, the effect continues for a longer period of time. Furthermore, when the proanthocyanidins contain at least 20 wt % of OPCs, a further superior effect of improving blood flow can be achieved.

The inventors of the present invention have reported that an effect of improving blood flow in the body is achieved by oral administration of proanthocyanidins (Japanese Patent Application No. 2002-219175). Since the proanthocyanidins have the property of constricting proteins, it is difficult to consider that when applied to the skin, the mucous membrane, and the like, the proanthocyanidins are absorbed topically into the body and improve blood flow. Therefore, the effect of the present invention would be unexpected.

Furthermore, when the external preparation for improving blood flow of the present invention contains a component other than the proanthocyanidins and that provides the effect of improving blood flow and/or a plant extract that does not contain proanthocyanidins and that provides the effect of improving blood flow, these substances can provide a synergistic effect of improving blood flow with the proanthocyanidins.

Examples of the component that provides the effect of improving blood flow include capsaicin and derivatives thereof; carpronium chloride; dialkyl monoamine derivatives; saponins; sinigrin; nicotinic acid and derivatives thereof and their salts; and γ-oryzanol. Capsaicine and derivatives thereof, saponins, and nicotinic acid and derivatives thereof are preferable.

Examples of the plant extract providing the effect of improving blood flow include capsicum extract, Panax ginseng extract, cresson extract, arnica extract, safflower extract, sophora root extract, and Japanese pepper extract. Capsicum extract, Panax ginseng extract, and sophora root extract are preferable.

In order for these components providing the effect of improving blood flow and/or plant extracts providing the effect of improving blood flow to achieve the synergistic effect of improving blood flow with the proanthocyanidins, it is preferable that they are contained in the external preparation for improving blood flow of the present invention in a ratio of 0.0001 wt % to 5 wt % and more preferably 0.001 wt % to 2 wt %.

The external preparation for improving blood flow of the present invention can be widely applied to drugs, quasi-drugs, cosmetics, toiletries, and the like. For example, skin lotions, facial creams, milky lotions, creams, packs, hair tonics, hair creams, shampoos, hair rinses, hair treatments, body shampoos, facial cleansers, soaps, foundations, face powders, lipsticks, lip glosses, rouges, eye shadows, hairdressings, hair restorers, water-based ointments, oil-based ointments, eye medicines, eyewashes, dentifrices, mouthwashes, fomentations, and gels are possible.

Moreover, for example, the external preparation for improving blood flow of the present invention can be contained in a food such as candy, gum, and gummi and can be applied to the inside of the oral cavity. Alternatively, the preparation can be contained in a carrier such as a fomentations and a gel or can be mixed with a crosslinking agent and can be applied topically. According to such applications, the rate of sustained release of the preparation can be controlled, and thus a long-acting administration can be performed.

### EXAMPLES

Hereinafter, the present invention will be described by way of examples. However, the present invention is not limited to these examples.

### Example 1

A skin lotion A having the composition shown in Table 1 below was prepared using an ethanol extract of pine bark (trade name: Flavangenol, produced by TOYO SHINYAKU Co., Ltd.) containing 40 wt % of proanthocyanidins (OPC content: 20 wt % in the extract) and 5 wt % of catechins, glycerin, citric acid, and sodium citrate.

### Examples 2 and 3

In Example 2, a skin lotion B having the composition shown in Table 1 was prepared by further adding a capsicum extract to the skin lotion of Example 1. In Example 3, a skin lotion C having the composition shown in Table 1 was prepared in the same manner as in Example 1 except that a previously prepared ethanol extract of pine bark containing 40 wt % of proanthocyanidins (OPC content: 5 wt % in the extract) and 5 wt % of catechins was used in place of the ethanol extract of pine bark in Example 1.

### Comparative Examples 1 to 3

In Comparative Example 1, a skin lotion D having the composition shown in Table 1 was prepared in the same manner as in Example 1 except that a swertia herb extract (Maruzen Pharmaceuticals Co., Ltd.) was used in place of the ethanol extract of pine bark in Example 1. In Comparative Example 2, a skin lotion E having the composition shown in Table 1 was prepared by further adding a capsicum extract to the skin lotion of Comparative Example 1. In Comparative Example 3, a skin lotion F having the composition shown in Table 1 was prepared in the same manner as in Example 1 except that water was used in place of the ethanol extract of pine bark in Example 1.

TABLE 1

| | | | | Comparative | | |
|---|---|---|---|---|---|---|
| | Examples | | | Examples | | |
| | 1 | 2 | 3 | 1 | 2 | 3 |
| Skin lotion | A | B | C | D | E | F |
| Pine bark extract | 0.01 | 0.01 | — | — | — | — |
| (OPC content: | | | | | | |
| 20 wt %) | | | | | | |
| Pine bark extract | — | — | 0.01 | — | — | — |
| (OPC content: 5 wt %) | | | | | | |
| Swertia herb extract | — | — | — | 0.01 | 0.01 | — |
| Capsicum extract | — | 0.001 | — | — | 0.001 | — |
| Glycerin | 0.001 | 0.001 | 0.001 | 0.001 | 0.001 | 0.001 |
| Citric acid | 0.05 | 0.05 | 0.05 | 0.05 | 0.05 | 0.05 |
| Sodium citrate | 0.04 | 0.04 | 0.04 | 0.04 | 0.04 | 0.04 |
| | | | | | | Unit: wt % |

The effect of improving blood flow was evaluated in the following manner using the obtained skin lotions A to F. First, a total of six marks each measuring 2.0 cm square were previously put on the forearm of each of ten subjects (healthy persons between the ages of 20 and 50), and the subcutaneous blood flow rate was measured in the areas of those marks using a rheometer (laser blood perfusion imager PIM II; Perimed AB, Sweden) and the resultant value was determined as a blood flow rate before application of the skin lotions. The average value of the blood flow rate before application of the skin lotions was taken as “a”.

Next, 0.1 ml of the skin lotions A to F were applied to the areas of the above-mentioned marks, respectively. Then, the subcutaneous blood flow rate was measured 0, 0.5, 1, 1.5, and 2 hours after the application. The average values of the obtained blood flow rate measured at predetermined times after the application of the skin lotions were taken as “b”.

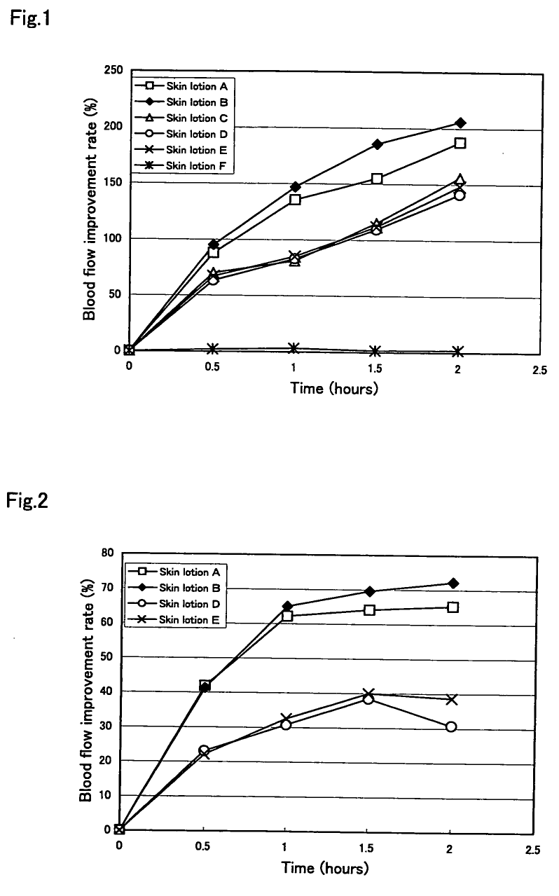

Based on the obtained average values a and b, the blood flow improvement rate was calculated using the following formula to evaluate the effect of improving blood flow:
Blood flow improvement rate(%)=100×(b−a)/a
FIG. 1 shows the results.

Referring to the results in FIG. 1, it can be seen that the skin lotions containing proanthocyanidins provide higher effects of improving blood flow, and the effects can be retained for a long period of time compared with those of the other skin lotions. Furthermore, a comparison between the skin lotions A and C shows that the skin lotion containing the proanthocyanidins having the higher OPC content provides a better effect of improving blood flow. Moreover, a comparison between the skin lotions B and E shows that when a component having the effect of improving blood flow other than the proanthocyanidins is contained in the skin lotion, a synergistic effect of improving blood flow derived from such component and the proanthocyanidins can be obtained.

Furthermore, the retention of the effect of improving blood flow was evaluated in the following manner using the skin lotions A, B, D, and E. First, a total of four marks each measuring 2.0 cm square were previously put on the forearm of each of ten subjects (healthy persons between the ages of 20 and 50), and the blood flow rate before application of the skin lotions was measured in the same manner as described above, and the average value a was calculated. Next, 0.03 ml, which was smaller than the amount described above, each of the skin lotions A, B, D, and E were applied, and the blood flow rate was measured at predetermined times after the application of the skin lotions in the same manner as described above, and the average values b were calculated. Blood flow improvement rate was calculated as mentioned above by using the average values a and b. FIG. 2 shows the results.

Referring to the results in FIG. 2, it can be seen that the effect of improving blood flow of the skin lotions containing proanthocyanidins continues for a long period of time even when the concentration is low. Furthermore, when a capsicum extract is contained in the skin lotions, the effect can continue even further.

### Example 4

A cream foundation having the components listed below was prepared using a pine bark extract (trade name: Flavangenol, produced by TOYO SHINYAKU Co., Ltd.):

<Components of the Foundation> (The Values are in wt %.)

| | |
|---|---|
| Pine bark extract | 0.05 |
| Triethanolamine | 1.2 |
| Sorbitol | 2.0 |
| Methyl parahydroxybenzoate | small quantity |
| Pigment (titanium oxide, talc, and kaolin) and fragrance | 15.5 |
| Stearic acid | 5.0 |
| Lipophilic glyceryl monostearate | 2.5 |
| Cetostearyl alchol | 1.0 |
| Propylene glycol monolaurate | 3.0 |
| Liquid paraffin | 7.0 |
| Isopropyl myristate | 8.0 |
| Butyl parahydroxybenzoate | small quantity |
| Water | 54.5 |

### Example 5

A lip cream having the components listed below was produced in the usual manner using the same pine bark extract as in Example 4:

<Components of the Lip Cream> (The Values are in wt %.)

| | |
|---|---|
| Pine bark extract | 1.0 |
| Ceresin | 4.0 |
| Candelilla wax | 8.0 |
| Carnauba wax | 2.0 |
| Isostearic acid diglyceride | 40.0 |
| Castor oil | 30.0 |
| Glycerin | 2.0 |
| Water | 13.0 |

Subjects using this lip cream remarked that the color or the luster of their lips was improved.

### Example 6

A hair tonic having the components listed below was produced using the same pine bark extract as in Example 4:

<Components of the Hair Tonic> (The Values are in wt %.)

| | |
|---|---|
| Pine bark extract | 0.1 |
| Sophora root extract | 0.1 |
| Glycerin | 3.0 |
| 1-Menthol | 0.2 |
| Polyoxyethylene hydrogenated castor oil | 0.2 |
| Ethanol | 60.0 |
| Water | 36.4 |

### Example 7

A mouth wash having the components listed below was produced in the usual manner using the same pine bark extract as in Example 4:

<Components of the Mouth Wash> (The Values are in wt %.)

| | |
|---|---|
| Pine bark extract | 0.01 |
| Panax ginseng extract | 0.1 |
| Ethyl alcohol | 15.0 |
| Saccharin sodium | 0.5 |
| Polyoxyethylene hydrogenated castor oil | 2.5 |
| Glycerin | 6.0 |
| Flavor | small quantity |
| Water | 75.5 |

### Example 8

A dentifrice having the components listed below was prepared in the usual manner using the same pine bark extract as in Example 4:

<Components of the Dentifrice> (The Values are in wt %.)

| | |
|---|---|
| Pine bark extract | 0.01 |
| Calcium carbonate | 39.0 |
| Sorbitol | 15.0 |
| Sodium carboxymethylcellulose | 0.6 |
| Sodium laurate | 1.3 |
| Saccharin | 0.09 |
| Mint flavor and preservative | small quantity |
| Water | 43.0 |

### Example 9

An ophthalmic solution (ophthalmic composition) having the components listed below was prepared in the usual manner using the same pine bark extract as in Example 4:

<Components of the Ophthalmic Solution> (The Values are in wt %.)

| | |
|---|---|
| Pine bark extract | 0.01 |
| Tear components | |
| Sodium chloride | 0.7 |
| Potassium chloride | 0.2 |
| Anti-inflammatory agent | |
| Sodium azulene sulfonate | 0.02 |
| Tetrahydrozoline hydrochloride | 0.02 |
| Antihistaminic agent | |
| Chlorpheniramine maleate | 0.03 |
| Water | 99.02 |

The invention may be embodied in other forms without departing from the spirit or essential characteristics thereof. The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

1. An external preparation for improving blood flow, comprising a pine bark extract and a capsicum extract, wherein the pine bark extract comprises proanthocyanidins.

2. (canceled)

3. The external preparation for improving blood flow of claim 1, wherein the pine bark extract comprises at least 20 wt % of oligomeric proanthocyanidin.

4. (canceled)

5. (canceled)

6. (canceled)

7. (canceled)

8. (canceled)

9. The external preparation for improving blood flow of claim 1, wherein the proanthocyanidins and the capsicum extract are contained in the preparation in amounts from 0.0001 wt % to 5 wt %, respectively.

10. The external preparation for improving blood flow of claim 3, wherein the proanthocyanidins and the capsicum extract are contained in the preparation in amounts from 0.0001 wt % to 5 wt %, respectively.

11. A method for improving blood flow comprising,
applying the external preparation of claim 1 to skin or mucous membrane of a subject.
